(19) United States
(12) Patent Application Publication
(10) Pub. No.: US 2026/0060940 A1
(43) Pub. Date: Mar. 5, 2026

(54) NUCLEOTIDE-BINDING DOMAIN AND LEUCINE-RICH REPEAT PROTEIN 3 INFLAMMASOME INHIBITOR FOR TREATING INFLAMMATORY INTESTINAL DISEASES

(71) Applicants: University of Macau, Macau (CN); Zhuhai UM Science & Technology Research Institute, Zhuhai (CN); Guangxi University of Chinese Medicine, Nanning (CN)

(72) Inventors: Yitao WANG, Zhuhai (CN); Hua LUO, Macau (CN); Guohui ZHANG, Macau (CN); Jingqi WANG, Macau (CN); Chun YAO, Nanning (CN); Jian XIAO, Nanning (CN); Erwei HAO, Nanning (CN)

(21) Appl. No.: 19/016,039

(22) Filed: Jan. 10, 2025

(30) Foreign Application Priority Data
Aug. 30, 2024 (CN) 202411209076.5

(51) Int. Cl. A61K 31/12; A61K 9/00; A61P 1/04; A61P 29/00; A61P 35/00; A61P 37/06

(52) U.S. Cl. CPC A61K 31/12; A61K 9/0053; A61P 1/04; A61P 29/00; A61P 35/00; A61P 37/06

(57) ABSTRACT

The present invention provides a method of treating a disease mediated by NLRP3 inflammasomes in a subject in need thereof, comprising administering to the subject a therapeutically effective amount of compound 2′,4′-dihydroxychalcone. The compound 2′,4′-dihydroxychalcone inhibits the activation of NLRP3 inflammasomes and reduces the secretion level of pro-inflammatory factors by inhibiting the activation of Caspase-1 and the expression of NLRP3.

### CROSS-REFERENCE TO RELATED APPLICATIONS

This application claims priority to Chinese Patent Application No. 202411209076.5, filed on Aug. 30, 2024, the contents of which are hereby incorporated by reference.

### TECHNICAL FIELD

The present disclosure relates to the technical field of biomedicine, and in particular to a nucleotide-binding domain and leucine-rich repeat protein 3 (NLRP3) inflammasome inhibitor for treating inflammatory intestinal diseases.

### BACKGROUND

Jigucao is a dried whole plant of Abrus cantoniensis Hance (ACH), an authentic Chinese herbal medicine in Lingnan area of China. ACH has a long history of use and has the functions of calming the liver, clearing away heat and detoxifying, and may be made into herbal tea or cold soup to cat. Since 1960s, Chinese scholars began to study the chemical constituents of ACH. Modern pharmacological studies show that ACH has many biological activities, such as liver protection, anti-inflammatory, immunomodulation, anti-oxidation and anti-tumor, which are closely related to its rich chemical constituents. Combined with the study of phytochemical and pharmacological activities, it was found that the main active components were alkaloids, flavonoids, triterpenoids and others. Up to now, most of the studies on the activity of the total extract have not been analyzed by chemical components, and the main effective components are unknown, so it is difficult to deeply understand the pharmacodynamic mechanism, target, pathway and application possibility in other diseases.

Inflammasome is a complex protein structure in the body, which has the function of regulating inflammatory response. The general structure of inflammasome includes nucleotide-binding oligomerization domain like receptor protein (NLRP, leucine rich repeat and pyrin domain containing), Apostasy-associated speck-like protein (ASC protein, containing a caspase recruitment domain protein (CARD) and caspase-1. Inflammasome is considered as an inflammatory switch in the body. NLRP3 inflammasome is the most studied inflammatory corpuscle at present, and is a multi-protein complex, playing a key role in innate immunity, and being also the first line of defense against pathogens, belonging to the nucleotide-binding oligomerization domain like receptor (NLR) protein family. NLRP3 inflammasome plays an important role in many human diseases driven by inflammation. The imbalance of NLRP3 inflammasome may lead to excessive inflammation and is related to various diseases, including autoimmune and autoimmune diseases, metabolic diseases and cancer.

The present disclosure intends to separate and identify components with inhibitory effect on NLRP3 inflammasome from ACH, so as to provide technical support for developing NLRP3 inflammasome inhibitors and treating diseases related to NLRP3 inflammasome.

### SUMMARY

An objective of the present disclosure is to provide a NLRP3 inflammasome inhibitor and an application thereof in preparing medicines for treating inflammatory intestinal diseases, to solve the problems existing in the prior art. The NLRP3 inflammasome inhibitor may effectively inhibit the activation of NLRP3 inflammasomes and may effectively treat diseases related to NLRP3 inflammasomes.

To achieve the above objective, the present disclosure provides the following scheme.

The present disclosure describes the isolation of bioactive compounds that inhibit the action of NLRP3 inflammasome. The present disclosure also describes a method of inhibiting NLRP3 inflammasome activation, comprising contacting the NLRP3 inflammasome in vitro with a compound 2′,4′-dihydroxychalcone. Uses of the compound to inhibit the NLRP3 inflammasome, to stabilize and/or decrease inflammatory intestinal diseases in subjects and to prevent and/or treat inflammatory intestinal diseases in subjects are encompassed.

The present disclosure provides a method of treating a disease mediated by NLRP3 inflammasomes in a subject in need thereof, comprising administering to the subject a therapeutically effective amount of compound 2′,4′-dihydroxychalcone.

The present disclosure also provides a method of treating a disease mediated by 2′,4′-dihydroxychalcone for treating a disease related to NLRP3 inflammasomes by administering to the subject a therapeutically effective amount is 10 mg/kg to 60 mg/kg, wherein the step of administering is performed orally.

Further, the disease related to NLRP3 inflammasomes is an inflammatory intestinal disease.

Further, the inflammatory intestinal disease is ulcerative colitis or colon cancer.

The present disclosure also provides a medicine for treating diseases related to NLRP3 inflammasomes, and an active ingredient includes 2′,4′-dihydroxychalcone.

Further, the medicine also includes pharmaceutically acceptable excipients.

Further, a dosage form of the medicine is an oral dosage form.

The present disclosure discloses the following technical effects.

According to the present disclosure, a compound-2′,4′-dihydroxychalcone with the function of inhibiting the activation of NLRP3 inflammasomes is screened from ACH, which may inhibit the activation of NLRP3 inflammasomes and reduce the secretion level of pro-inflammatory factors by inhibiting the activation of caspase-1 and the expression of NLRP3. The compound plays an important role in regulating intestinal homeostasis and inhibiting colitis and colon cancer.

The NLRP3 inflammasome inhibitor provided by the present disclosure may effectively inhibit the activation of NLRP3 inflammasomes in vivo and in vitro and may effectively treat diseases related to NLRP3 inflammasomes. The present disclosure provides technical support for treating diseases related to NLRP3 inflammasomes.

The 2′,4′-dihydroxychalcone according to the present disclosure, as an effective component in ACH, is not only easy to obtain raw materials and cheap in price, but also helpful to expand the clinical application research of ACH.

### BRIEF DESCRIPTION OF THE DRAWINGS

To explain the embodiments of the present disclosure or the technical solution in the prior art more clearly, the drawings needed in the embodiments will be briefly introduced below. Apparently, the drawings in the following description are only some embodiments of the present disclosure. For one of ordinary skill in the art, other drawings may be obtained according to these drawings without paying creative labor.

FIG. 1A is a statistical chart of THP-1 cell viability in different treatment groups.

FIG. 1B is a statistical chart of J774A.1 cell viability in different treatment groups.

FIG. 1C is a statistical chart of interleukin-1 beta (IL-1β) secretion in THP-1 cells of different treatment groups.

FIG. 1D is a statistical chart of IL-1β secretion in J774A.1 cells of different treatment groups, where ***P<0.001, ns stands for no significant difference.

FIG. 2 is a statistical chart of tumor necrosis factor-α (TNF-α) secretion in J774A.1 cells of different treatment groups, where ns stands for no significant difference.

FIG. 3 shows Western blotting detection results of J774A.1 cells in different treatment groups.

FIG. 4A shows Western blotting detection results of HT29 cells in different treatment groups.

FIG. 4B shows Western blotting detection results of CT26 cells in different treatment groups.

FIG. 5A is a diagram of the weight change of mice in different experimental groups in Embodiment 2.

FIG. 5B is a diagram of the disease activity index (DAI) of mice in different experimental groups in Embodiment 2.

FIG. 5C is a diagram of the colon length of mice in different experimental groups in Embodiment 2.

FIG. 5D is a colon representative diagram of mice in different experimental groups in Embodiment 2, where n=10, compared with dextran sulfate sodium (DSS) group, ***P<0.001.

FIG. 6 is a hematoxylin-eosin staining diagram of colon tissues of mice in different experimental groups in Embodiment 2.

FIG. 7A is a statistical chart of the contents of colon inflammatory factor IL-1β in different treatment groups in Embodiment 2.

FIG. 7B is a statistical chart of the contents of colon inflammatory factor interleukin-6 (IL-6) in different treatment groups in Embodiment 2.

FIG. 7C is a statistical chart of contents of colon inflammatory factor TNF-α in different treatment groups in Embodiment 2, where compared with the DSS group, ***P<0.001.

FIG. 8 shows Western blotting detection results of mice in different experimental groups in Embodiment 2.

FIG. 9A shows the tumor growth curve of mice in different experimental groups in Embodiment 3.

FIG. 9B shows weight change of mice in different experimental groups in Embodiment 3.

FIG. 9C is a tumor weight statistical chart of mice in different experimental groups in Embodiment 3, where n=10, *P<0.05, **P<0.01, ***P<0.001.

FIG. 10 is a staining diagram of Ki67 in HT29 tumor tissues of mice in different experimental groups in Embodiment 3.

FIG. 11 is a staining diagram of NLRP3 in HT29 tumor tissues of mice in different experimental groups in Embodiment 3.

FIG. 12 shows Western blotting detection results of mice in different experimental groups in Embodiment 3.

FIG. 13A is a statistical chart of the liver index of mice in different experimental groups in Embodiment 3.

FIG. 13B is a statistical chart of the spleen index of mice in different experimental groups in Embodiment 3, where n=10, *P<0.05, **P<0.01, ***P<0.001.

### DETAILED DESCRIPTION OF THE EMBODIMENTS

A number of exemplary embodiments of the present disclosure will now be described in detail, and this detailed description should not be considered as a limitation of the present disclosure but should be understood as a more detailed description of certain aspects, characteristics and embodiments of the present disclosure.

It should be understood that the terminology described in the present disclosure is only for describing specific embodiments and is not used for limiting the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges may be independently included or excluded from the range.

Unless otherwise specified, all technical and scientific terms used herein have the same meaning as commonly understood by one of ordinary skill in the art to which the present disclosure relates. Although the present disclosure only describes the preferred methods and materials, any methods and materials similar or equivalent to those described herein may also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the present disclosure. The description and embodiments of that present disclosure are exemplary only.

The terms “comprising”, “including”, “having” and “containing” used herein are all open terms, which means including but not limited to.

### Description of Terms

PMA refers to phorbol-12-myristate-13-acetate;
LPS refers to lipopolysaccharide;
DSS refers to dextran sulfate sodium.

According to the present disclosure, a compound with an inhibitory effect on an NLRP3 inflammasome is isolated from Abrus cantoniensis Hance, and is identified as 2′,4′-dihydroxychalcone, and the structural formula is as follows:

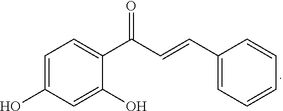 [SMILES: O=C(/C=C/c1ccccc1)c1ccc(O)cc1O]

### Embodiment 1 Study on Inhibitory Activity of 2′,4′-Dihydroxychalcone Compound on NLRP3 Inflammasomes in Macrophages

### 1. Experimental Method

### (1) Cytotoxicity Study

THP-1 and J774A.1 cells induced by LPS were inoculated into 96-well plates, and incubated respectively in a constant temperature incubator at 37° C. at 5% carbon dioxide concentration. After the cells adhered to the wall, 2′,4′-dihydroxychalcone with different concentrations (5, 10, 20 micromole (μM)) was added and incubated for 2 hours (h), respectively, and the cell viability was measured by cell counting kit-8 (CCK-8) method.

### (2) Study on Inhibiting Activity of NLRP3 Inflammasomes of Macrophages

THP-1 cells were plated into a 6-well plate, treated with PMA (10 nanogram per milliliter (ng/mL)) overnight, then treated with LPS (500 ng/ml) for 3 h, then treated with different concentrations of 2′,4′-dihydroxychalcone (5, 10 and 20 μM) for 1 h, and then incubated with Nigericin (10 μM, 1 h).

J774A.1 cells were plated into a 6-well plate, incubated overnight with LPS (1 microgram per milliliter (μg/mL)) after 24 h, then incubated with different concentrations of 2′,4′-dihydroxychalcone (5, 10 and 20 μM) for 1 h, and then incubated with Nigericin (10 μM, 1 h).

The supernatant of each treatment group was collected and detected by enzyme linked immunosorbent assay (ELISA), and the cells were collected for protein Western blotting analysis.

### (3) Study on Inhibiting Activity of NLRP3 Inflammasomes in Colon Cancer Cell Lines

HT29 and CT26 cells were inoculated into 6-well plates, and treated with different doses of 2′,4′-dihydroxychalcone (5, 10 and 20 μM) for 24 hours after adherence. Bicinchoninic acid (BCA) quantitative analysis was performed after cell lysis. The lysate was mixed with 5× loading buffer, separated by 8% sodium dodecyl sulfate polyacrylamide gel electrophoresis (SDS-PAGE), and transferred to polyvinylidene fluoride (PVDF) membrane. After blocking for 1 hour at room temperature, PVDF membrane was incubated with specific primary antibody at 4° C. overnight. Washing was performed with the tris-buffered saline with tween-20 (TBST) buffer for 5 times, the secondary antibody was added and incubated at room temperature for 1 h. After washing for 5 times, enhanced chemiluminescence (ECL) reagent showed protein bands.

### 2. Experimental Results

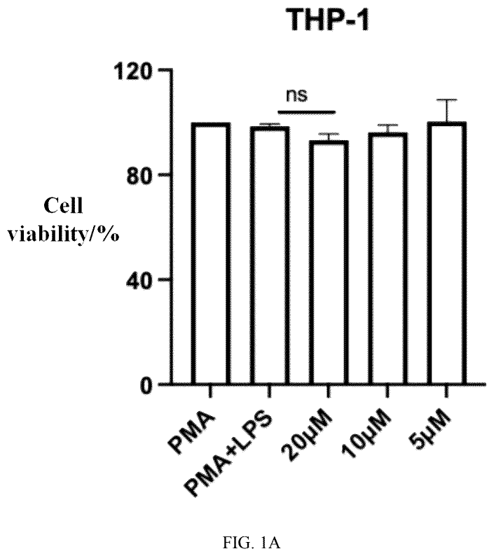

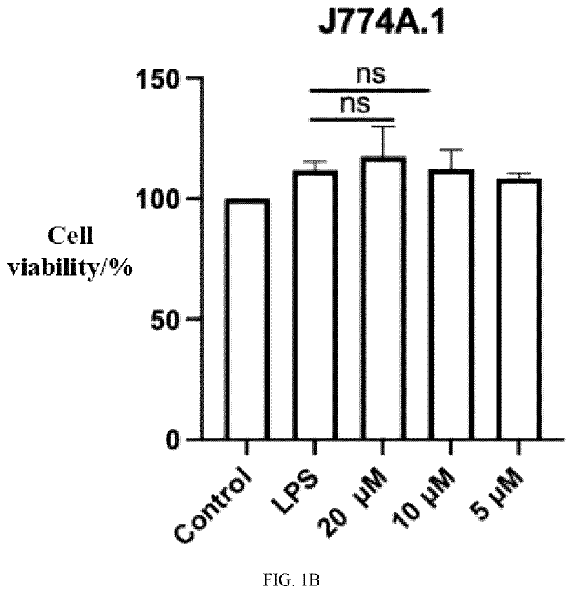

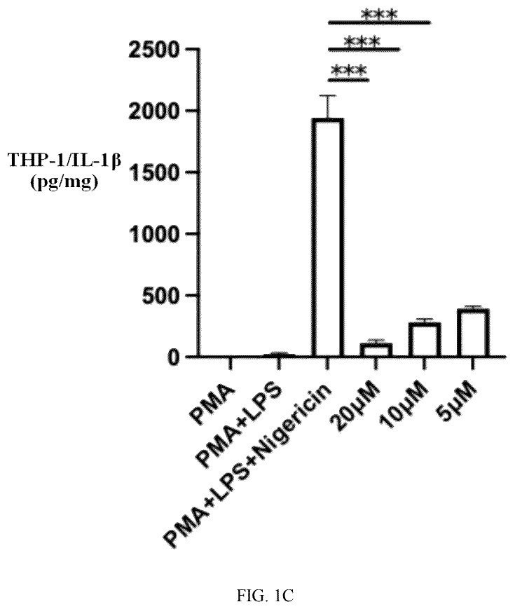

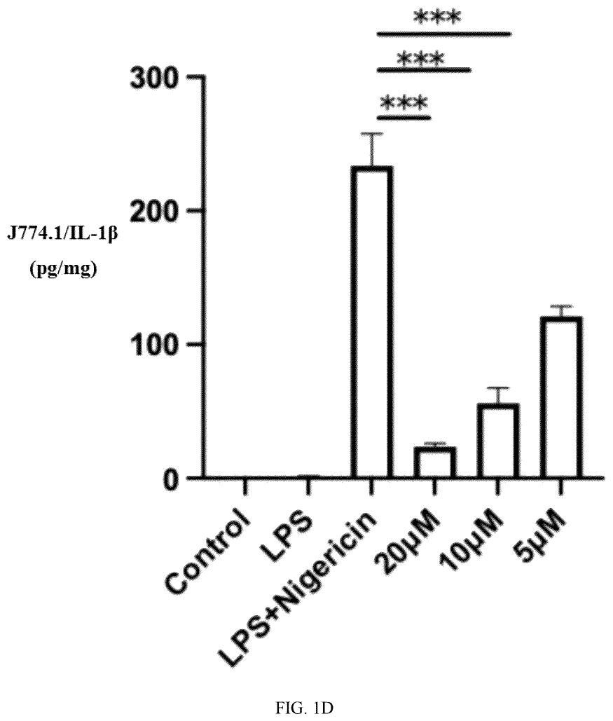

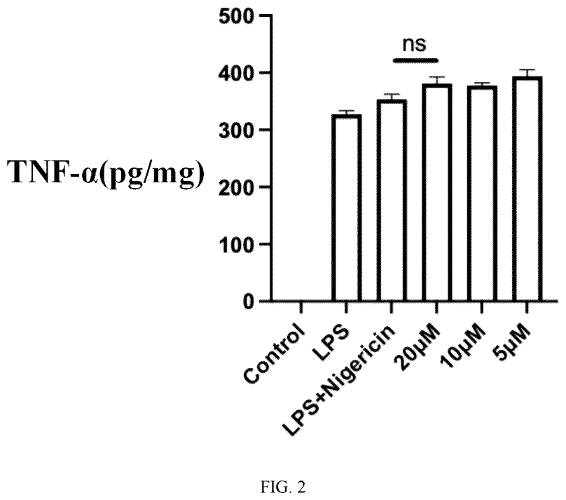

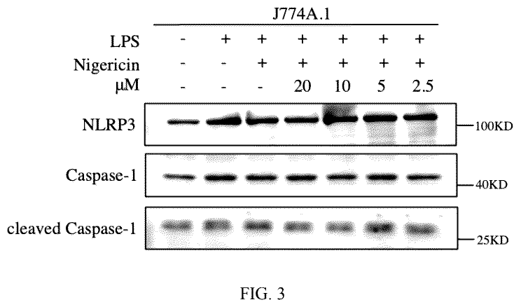

It may be seen from FIG. 1A and FIG. 1B that when the concentration of 2′,4′-dihydroxychalcone is 20 μM, the cell viability of THP-1 and J774A.1 cells are close to 100%, indicating that the compound has good safety. As shown in FIG. 1C and FIG. 1D, 2′,4′-dihydroxychalcone inhibits the secretion of IL-1β in a dose-dependent manner. As shown in FIG. 2, 2′,4′-dihydroxychalcone does not inhibit the secretion of TNF-α that is not associated with the activation of inflammasomes. As shown in FIG. 3, 2′,4′-dihydroxychalcone may inhibit the activation of Caspase-1 in J774A.1 macrophages, indicating that 2′,4′-dihydroxychalcone may inhibit the activation of NLRP3 inflammasomes by inhibiting caspase-1 cleavage.

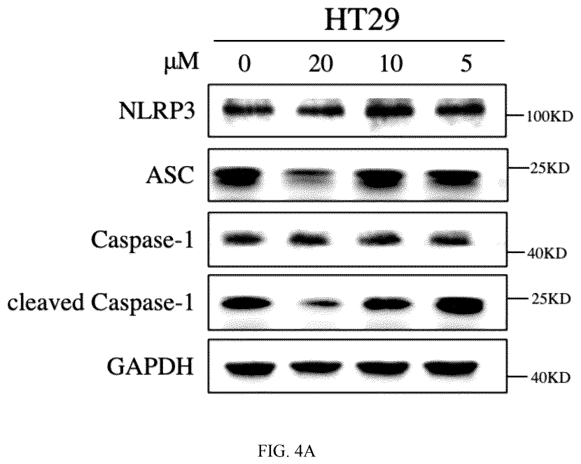

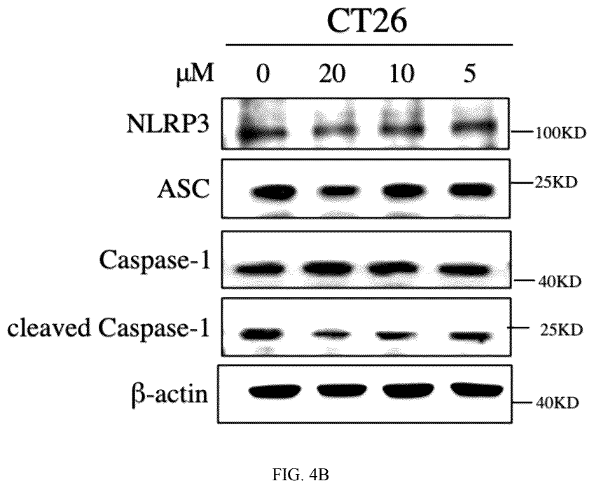

As may be seen from FIG. 4A and FIG. 4B, 2′,4′-dihydroxychalcone may directly inhibit the expression of NLRP3, Caspase-1 and ASC in colon cancer cells, and may also inhibit the activation of caspase-1, thus directly inhibiting the activation of NLRP3 inflammasomes.

Taking colitis and colon cancer as examples, the effect of 2′,4′-dihydroxychalcone in the treatment of diseases related to NLRP3 inflammasomes is explained below.

### Embodiment 2 Study on Efficacy of Improving DSS-Induced Ulcerative Colitis with Compounds

### 1. Experimental Method

### (1) Establishment and Pharmacodynamic Evaluation of a Model of DSS-Induced Acute Colitis in Mice.

Forty specific pathogen free (SPF) male mice C57BL/6J aged 6-8 weeks were housed adaptively for 7 days, and the mice were randomly divided into four groups (10 mice in each group): control group (Control group), model group (DSS group) and two treatment groups, where the two treatment groups were low dose group (10 milligram per kilogram (mg/kg)) and high dose group (30 mg/kg) respectively. DSS group was given 2.5% DSS (weight/volume (w/v)) aqueous solution orally for 7 days (4th-10th day). The low dose and high dose groups of the compound were given different doses of 2′,4′-dihydroxychalcone (0.4% sodium carboxymethyl cellulose as solvent) by intragastric administration from first day to 10th day, and were given 2.5% DSS (w/v) aqueous solution orally for 7 days (4th-10th day). The control group was not given 2′,4′-dihydroxychalcone and DSS, but was replaced by the same amount of solvent.

The weight of mice was recorded every day, and the disease activity index (DAI) was scored according to the weight loss, stool viscosity and stool bleeding. The score of each indicator ranged from 0 to 4. The mice were killed on the 11th day, and the colon tissues were collected for analysis.

### (2) Histopathological Analysis

Histological analysis of colon tissues was carried out by hematoxylin and eosin (H&E) staining. The fixed colons were embedded in paraffin before being used, stained with hematoxylin-eosin, dehydrated and dried, sealed with neutral gum, and then observed by microscope.

### (3) Determination of Inflammatory Factors in Colon Tissues

After grinding the colon tissues, the supernatant was centrifuged for ELISA determination and BCA protein quantification. According to the instructions of ELISA kit, detection and content analysis were performed to the inflammatory factors TNF-α, IL-6 and IL-1β.

### (4) Analysis of Protein Expression of NLRP3 Inflammasomes

Radio immunoprecipitation assay (RIPA) lysis buffer was used to lyse colon tissues. After quantitative analysis by BCA, the lysate was mixed with 5× loading buffer, separated by 8% SDS-PAGE and transferred to PVDF membrane. After blocking for 1 h at room temperature, PVDF membrane was incubated with specific primary antibody at 4° C. overnight. Washing was performed with the TBST buffer for 5 times, the secondary antibody was added and incubated at room temperature for 1 h. After washing for 5 times, ECL reagent showed protein bands.

### 2. Experimental Results

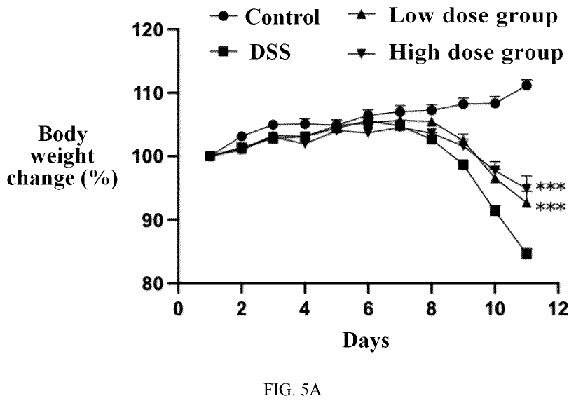

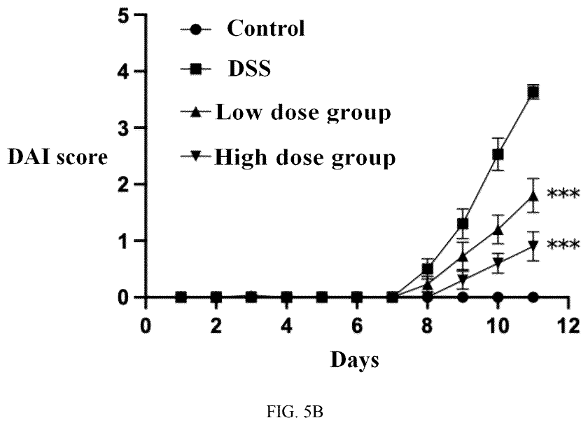

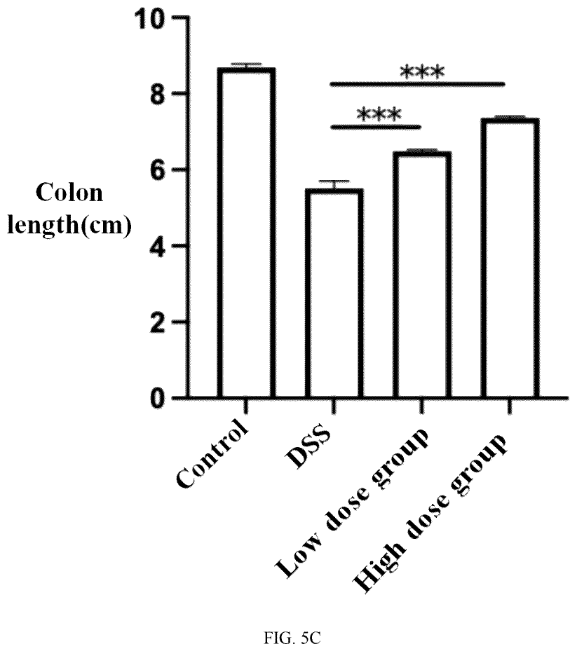

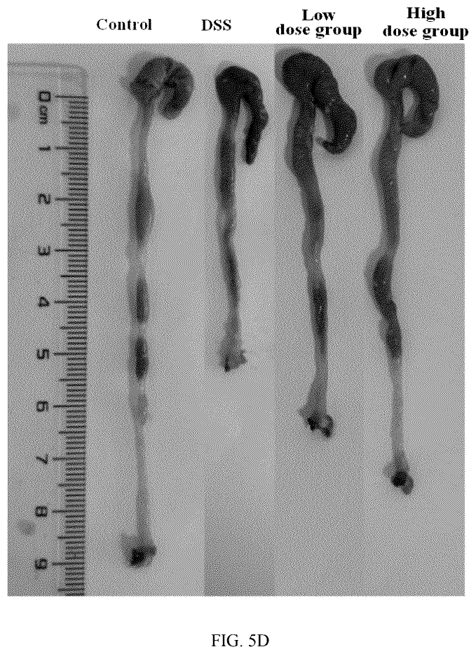

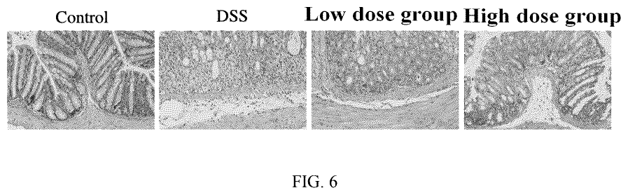

The results showed that compared with the mice in the Control group, after DSS-induced ulcerative colitis was adopted, the weights of the mice in the model group and the two treatment groups gradually decreased (FIG. 5A), and the disease activity index DAI scores increased (FIG. 5B), and the colon lengths were significantly shortened (FIG. 5C), and FIG. 5D was a representative colon diagram. The 2′,4′-dihydroxychalcone administration group increased the colon length of mice in DSS group and decreased the DAI score in a dose-dependent manner. Pathological evaluation of colon tissues was made by H&E staining. The staining results were shown in FIG. 6. The results showed that the overall structure of intestinal tissue in mice with DSS-induced colitis was abnormal, local mucosal necrosis was obvious, and the normal structure of intestinal tissue basically disappeared. There were a large number of inflammatory cells in the whole tissue, and there were dense areas of inflammatory cell aggregation. In the treatment group, the histological microstructure of H&E staining sections was improved, the infiltration of inflammatory cells was reduced, and there was no area where inflammatory cells gathered, which could alleviate the morphological changes of colonic recess in DSS group and protect goblet cells. Thus, the NLRP3 inflammasome inhibitor-2′,4′-dihydroxychalcone according to the present disclosure could effectively improve the pathological injury of colon in mice.

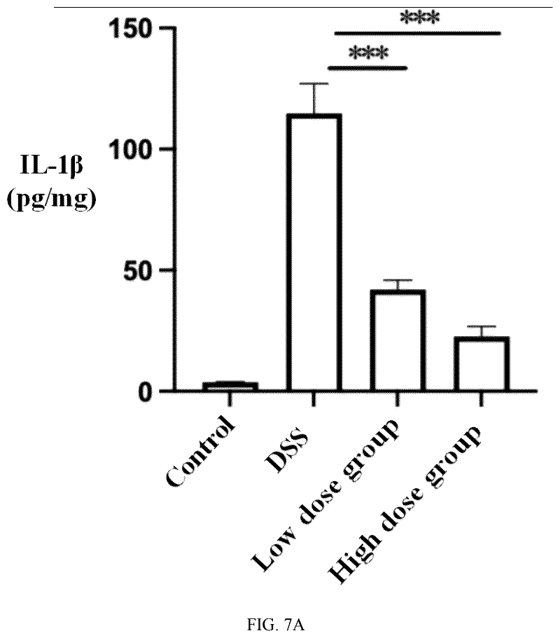

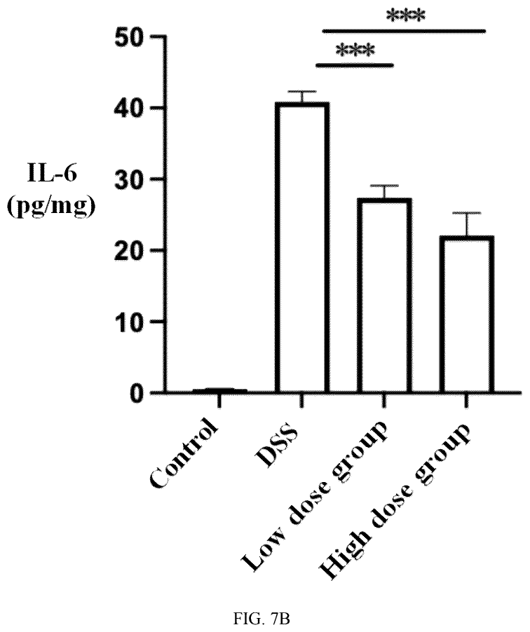

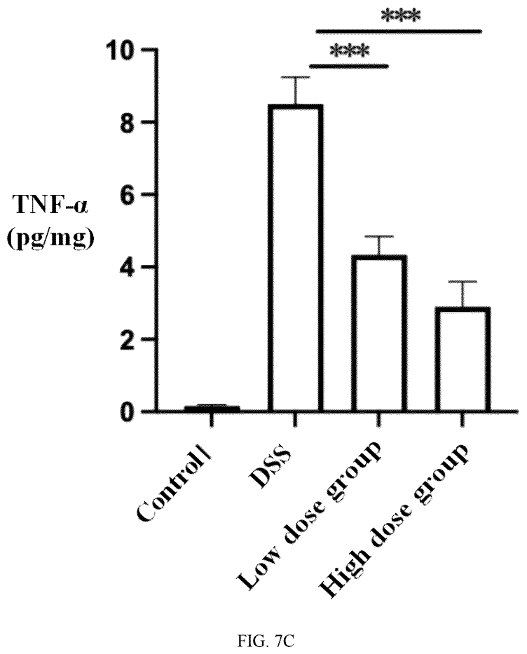

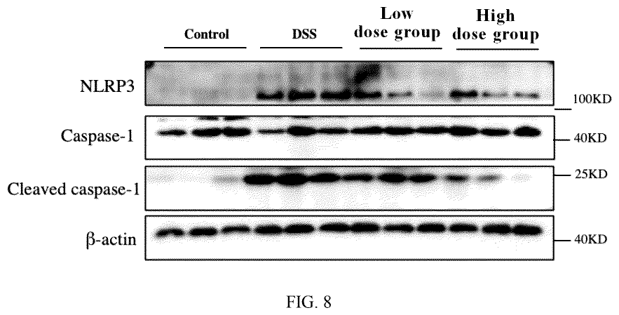

In addition, as shown in FIG. 7A-FIG. 7C, the levels of pro-inflammatory factors IL-6, IL-1β and TNF-α in colon tissues were significantly reduced after treatment with the NLRP3 inflammasome inhibitor-2′,4′-dihydroxychalcone according to the present disclosure. Western blotting analysis showed that 2′,4′-dihydroxychalcone could inhibit the expression of NLRP3 protein and the activation of caspase-1 in colon (FIG. 8).

To sum up, the NLRP3 inflammasome inhibitor-2′,4′-dihydroxychalcone according to the present disclosure may significantly reduce the level of pro-inflammatory factors and inhibit the activation of NLRP3 inflammasomes, so as to alleviate the intestinal inflammation of DSS-induced ulcerative colitis in mice.

### Embodiment 3 Study on Pharmacodynamics of Compounds in Inhibiting Growth of HT29 Tumor In Vivo

### 1. Experimental Method

### (1) Establishment and Pharmacodynamic Evaluation of HT29 Human Colon Cancer Model

Thirty SPF BALB/c-Nude female rats aged 6-8 weeks were reared adaptively for 7 days. HT29 cells with 8×10⁶ cells/100 μL were injected subcutaneously into the right armpit of BALB/c nude mice. After the obvious tumors were observed, the mice were divided into three groups (10 mice in each group): model group (Model), low dose group (30 mg/kg) and high dose group (60 mg/kg). After grouping, the low dose group and the high dose group were given 2′,4′-dihydroxychalcone by gavage every day according to their respective doses until the end of the experiment. After 13 days of administration, the mice were killed, and the tumors were weighed and photographed. Some tumors were stored at −80° C. for Western blotting and other analysis, while others were stored with 4% paraformaldehyde for Immunohistochemistry (IHC) and other detection.

Tumor growth inhibition rate: tumor size and mouse weight were monitored every 2 days. The tumor volume was measured with vernier caliper. The long side and short side of the tumor were taken as length (a) and width (b) respectively, and the volume=a×b×b/2, and the tumor growth curve was drawn. Tumor inhibition rate (%)=(average tumor weight in model group-average tumor weight in administration group)/average tumor weight in model group×100%.

### (2) Histopathological Analysis

The fixed tumor tissue was embedded in paraffin before being used. In IHC experiment, anti-NLRP3 antibody (GB114320, Servicebio), anti-Ki67 antibody (GB 11141, Servicebio) and horseradish peroxidase (HRP) labeled anti-rabbit immunoglobulin G (IgG) (AB205718, Abcam) were used to incubate tissue sections, and hematoxylin and diaminobenzidine (DAB) staining was used. The final stained sections were observed under a microscope system (Nikon Eclipse CI, Nikon).

### (3) Analysis of Protein Expression of NLRP3 Inflammasomes

RIPA lysis buffer was used to lyse tumor tissue. After quantitative analysis by BCA, the lysate was mixed with 5× loading buffer, separated by 8% SDS-PAGE and transferred to PVDF membrane. After blocking for 1 h at room temperature, PVDF membrane was incubated with specific primary antibody at 4° C. overnight. Washing was performed with the TBST buffer for 5 times, the secondary antibody was added and incubated at room temperature for 1 h. After washing for 5 times, ECL reagent showed protein bands.

### (4) Organ Index Analysis

After dissection, the liver and spleen, the main immune organs, were weighed and recorded. Organ index=organ weight/weight×100%.

### 2. Experimental Results

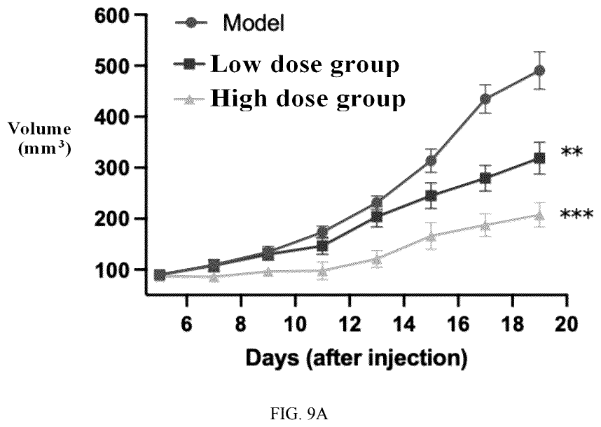

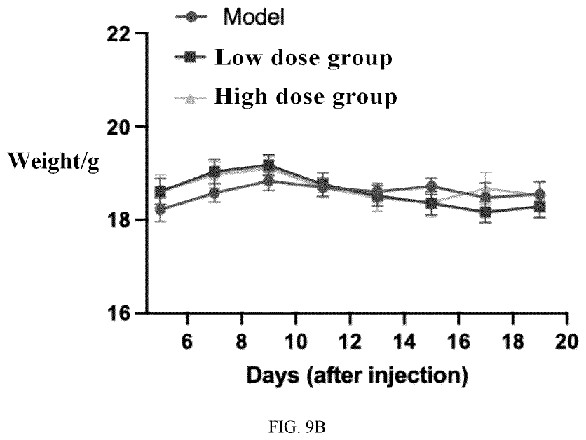

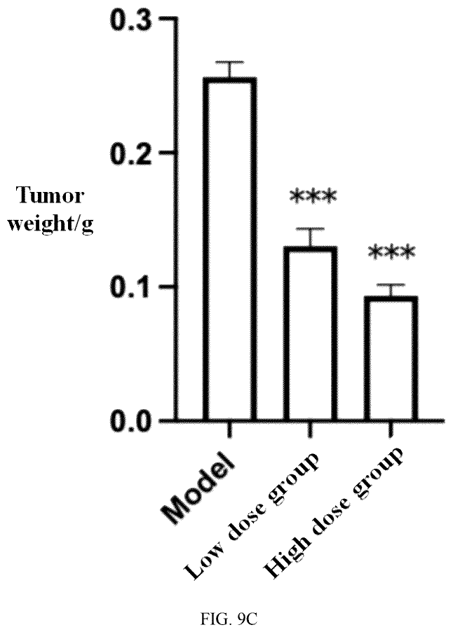

As shown in FIG. 9A-FIG. 9B, 2′,4′-dihydroxychalcone may significantly inhibit the growth of HT29 transplanted tumor (FIG. 9A) without affecting the weight of mice (FIG. 9B). Based on the tumor weight (FIG. 9C), the tumor inhibition rates of low dose group and high dose group are 49.2% and 61.0% respectively.

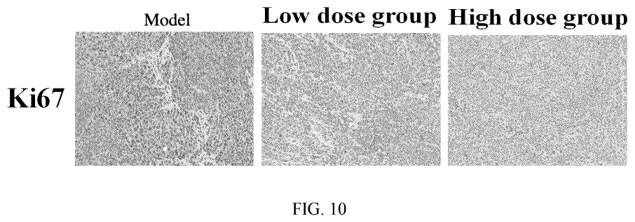

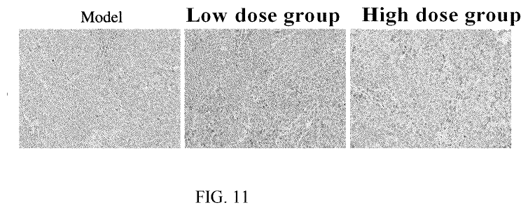

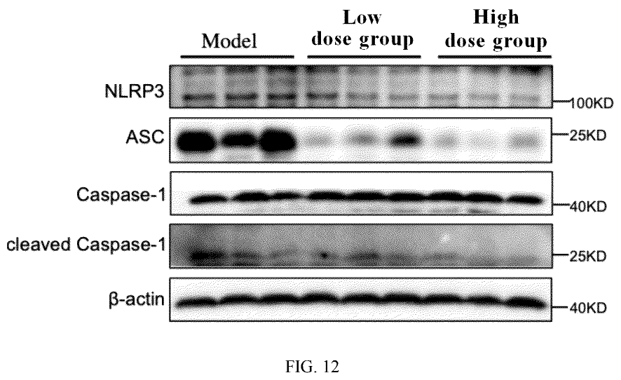

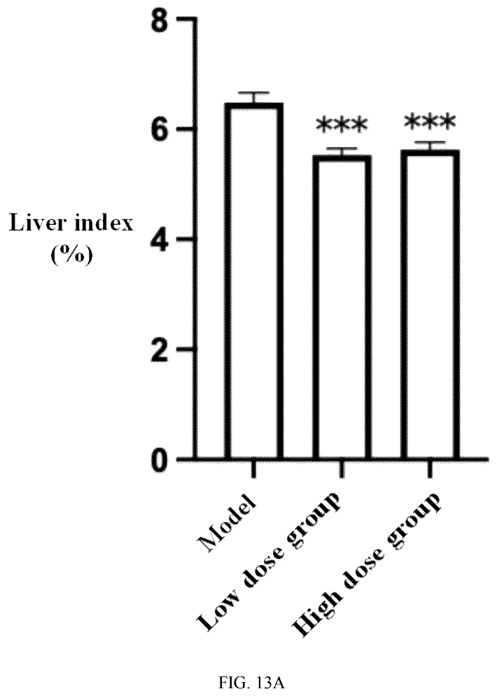

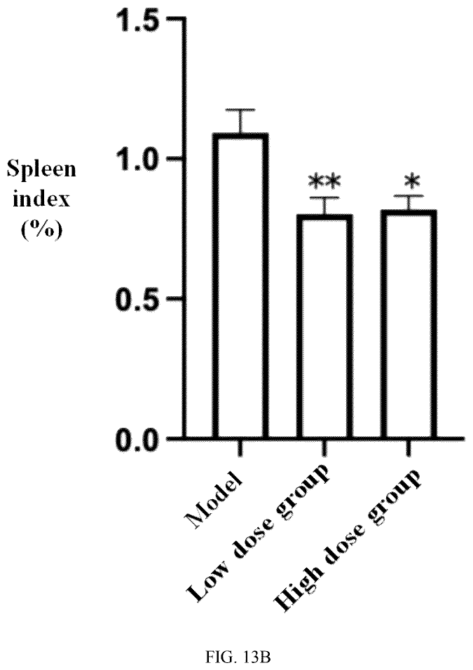

2′,4′-dihydroxychalcone may significantly reduce the expression levels of Ki67 (FIG. 10) and NLRP3 (FIG. 11) in HT29 tumor tissues, indicating that 2′,4′-dihydroxychalcone may inhibit the proliferation of tumor cells and the expression of NLRP3 in tumor. Western-blotting results also show that 2′,4′-dihydroxychalcone may significantly inhibit the expression of NLRP3 and ASC and the activation of caspase-1 (FIG. 12). In addition, 2′,4′-dihydroxychalcone may also reduce the liver index (FIG. 13A) and spleen index (FIG. 13B) of tumor-bearing mice to normal levels, indicating that 2′,4′-dihydroxychalcone may reduce the tumor load of BALB/c-Nude mice.

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical solution of the present disclosure should fall within the protection scope of the present disclosure.

What is claimed is:

1. A method of inhibiting NLRP3 inflammasome activation, comprising: contacting the NLRP3 inflammasome in vitro with a compound 2′,4′-dihydroxychalcone.

2. A method of treating a disease mediated by NLRP3 inflammasomes in a subject in need thereof, comprising administering to the subject a therapeutically effective amount of compound 2′,4′-dihydroxychalcone.

3. The method according to claim 2, wherein the disease is an inflammatory intestinal disease.

4. The method according to claim 3, wherein the inflammatory intestinal disease is ulcerative colitis or colon cancer.

5. The method of claim 2, wherein the therapeutically effective amount is 10 mg/kg to 60 mg/kg.

6. The method of claim 2, wherein the step of administering is performed orally.
